(19) United States
(12) Patent Application Publication
(10) Pub. No.: US 2014/0329707 A1
(43) Pub. Date: Nov. 6, 2014

(54) NANOSCALE COAXIAL ELECTRODE ARRAYS AND METHODS THEREOF

(71) Applicant: The Trustees of Boston College, Chestnut Hill, MA (US)

(72) Inventors: Michael J. Naughton, Newton, MA (US); Thomas C. Chiles, Chestnut Hill, MA (US); Michael J. Burns, Chestnut Hill, MA (US); John P. Christianson, Chestnut Hill, MA (US); Jeffrey R. Naughton, Chestnut Hill, MA (US); Binod Rizal, Chestnut Hill, MA (US)

(21) Appl. No.: 14/269,986

(22) Filed: May 5, 2014

Related U.S. Application Data
(60) Provisional application No. 61/819,616, filed on May 5, 2013.

(51) Int. Cl. G01N 33/94; A61B 5/04; A61N 5/06; G01N 33/487

(52) U.S. Cl. CPC G01N 33/9406; G01N 33/48728; A61B 5/04001; A61N 5/0622
USPC 506/9; 506/39; 435/288.7; 435/29; 600/300

(57) ABSTRACT

The invention provides novel devices and methods that enable ultrahigh spatial and temporal resolution interfaces that allow access and intervention to local (intra- and proximate extra-neuronal) neuroelectronic and neurotransmitter molecular signatures associated with aberrant cell function and cell death leading to neurodegenerative diseases. Scalable devices based on a unique nanocoaxial electrode array of the invention offer neural recording and control at unprecedented levels of precisions.

### PRIORITY CLAIMS AND RELATED APPLICATIONS

This application claims the benefit of U.S. Provisional Application No. 61/819,616, filed May 5, 2013, the entire content of which is incorporated herein by reference in its entirety.

### TECHNICAL FIELDS OF THE INVENTION

The invention generally relates to novel devices and methods for cellular and biological measurement and analysis. More particularly, the invention relates to novel devices providing a neuroelectronic/optogenetic interface with ultrahigh spatial resolution, which is suitable for in vitro or in vivo stimulation and recording of neuronal activity such as brain function.

### BACKGROUND OF THE INVENTION

The central nervous system, made up of the brain and the spinal cord, controls all the workings of the human body. Structural, biochemical or electrical abnormalities in the nervous system can result in a range of conditions and diseases. Symptoms can include difficulty with motion, speaking, swallowing, breathing, learning, memory, and emotion regulation. Degenerative diseases, such as Parkinson's and Alzheimer's, can result from damage to or death of nerve cells. Faulty genes can also cause nervous system disorders, such as Huntington's disease and muscular dystrophy. The World Health Organization estimates that as many as one billion people worldwide are affected by neurological disorders.

Devices and methods that exert influence on or allow manipulation of electrical activities of neurons, especially of neural ensembles or networks, can lead to effective intervention and treatment of neurological diseases, such as epilepsy, Alzheimer's and Parkinson's, as well as providing tools for research into these conditions. Concurrent measurements of the electrical activities of large numbers of neurons with deep subcellular resolution can provide valuable insights into a wide range of neural phenomena that intrinsically emerge at the network level.

Intraneural and interneural processes, for example, recording and manipulation of synaptic terminals or presynaptic membrane potentials, require both electrical and spatial precisions which state of the art technologies are unable to provide. Currently, there is a mismatch between the size scales of neuroelectronic interface technologies and those of neurons. Existing electrophysiological tools, such as patch clamps, sharp electrodes and microelectrode arrays (MEA), have limited spatial resolution, and are thus unable to probe important details of intra- and intercellular communication. These tools do not provide nanoscale (sub-micrometer) resolution or precision. Instead, one has to choose between electrical precision (patch clamp) or multiplexing with high spatial resolution (microelectrode arrays) but not both. Significantly, the spatial resolution of commercially available MEAs is quite limited relative to the dimensions of neurons.

In light of recent advances in the area of optogenetics, incorporating light to neural interface devices has become increasingly significant. Optogenetic tools permit control over the biochemical and electrical properties of individual neurons with photons, affording unprecedented temporal and cellular (genotype) accuracy of neural control. Yet numerous physical limitations impede the development of optogenetic devices, so-called “optrodes”, and state of the art embodiments lack the spatial resolution needed to control individual neurons and synapses.

Thus, a critical unmet need remains for novel devices and methods that are able to provide interfaces of ultrahigh spatial and temporal resolutions, enabling access and intervention to local (intra- and proximate extra-neuronal) neuroelectronic and neurotransmitter molecular signatures associated with aberrant cell function and cell death leading to neurodegenerative diseases.

### SUMMARY OF THE INVENTION

The invention provides novel devices and methods that enable interfaces of ultrahigh spatial and temporal resolutions and provide both access and intervention to intra- and proximate extra-neuronal neuroelectronic and neurotransmitter molecular activities. Importantly, the invention offers a scalable solution to neural recording and control founded on a unique nanocoaxial electrode architecture. Devices disclosed herein are based on next-generation neuroelectronic and optrode arrays that move beyond the spatial resolution of existing MEAs while uniquely incorporating simultaneous light delivery and electronic recording in a single device element by integrating optical delivery, and electrical sensing of neuron activity (e.g., spikes, action potentials, neurotransmitter release). Thus, the invention offers a new neurosensing technology capable of interrogating and dynamically controlling neuronal activities with unprecedented resolution and precision, giving rise to novel approaches for treatments of neurological diseases.

In one aspect, the invention generally relates to a nanocoaxial electrode. The nanocoaxial electrode includes: a conductive inner core; a coaxial dielectric layer surrounding the conductive inner core; and a coaxial conductive outer layer encasing the dielectric layer. The coaxial conductive outer layer is adapted to electronically and electromagnetically shield the conductive core, and the diameter of the coaxial conductive outer layer is less than about 1 μm.

In certain embodiments of the nanocoaxial electrode, the conductive inner core and conductive outer layer are adapted to serve as recording and reference electrodes. In certain embodiments, the nanocoaxial electrode further includes an embedded metal nanowire optical antenna. In certain embodiments, the coaxial structure is adapted to serve as an optical light guide, with light propagating along or emanating from the dielectric layer. In certain embodiments, the coaxial dielectric layer is adapted to serve as a light emitting diode providing spatially discrete illumination. In certain embodiments, the coaxial dielectric layer is adapted to serve as an electroluminescent component providing spatially discrete illumination.

In another aspect, the invention generally relates to a nanocoaxial optrode array. The nanocoaxial optrode array includes: a plurality of inter-connected nanocoaxial electrodes arranged in an array; a light delivery component; and an electronic recording component. The nanocoaxial electrode includes a conductive inner core; a coaxial dielectric layer surrounding the conductive inner core; and a coaxial conductive outer layer encasing the dielectric layer, and wherein the diameter of the coaxial conductive outer layer is less than about 1 μm.

In certain embodiments, the nanocoaxial optrode array is coupled to a light emitting diode (LED) array.

In another aspect, the invention generally relates to a method for detecting extracellular neuronal activity. The method includes: providing a neuroelectronic probe comprising one or more nanocoaxial optrode arrays; contacting the neuroelectronic probe with a tissue sample or other neuron source; and manipulating the neuroelectronic probe to electrically detect extracellular neuronal activity of the tissue sample.

In certain embodiments, the method further includes recording extracellular neuronal activity of the tissue sample. In certain embodiments, the method is performed in vitro. In certain embodiments, the method is performed in vivo.

### BRIEF DESCRIPTION OF THE FIGURES

FIG. 1 depicts a schematic showing a side view of an exemplary embodiment of nanocoaxial electrodes.

FIG. 2 depicts a schematic showing a top view of an exemplary embodiment of an array of nanocoaxial electrodes.

FIG. 3 depicts a perspective view of an embodiment of an array of nanocoaxial electrodes with inner and outer conductors exposed and a protruded inner conductor.

FIG. 4 depicts schematic representations of the nanocoaxial architecture as a high-resolution optrode for neuroelectronic and optogenetic characterization and control.

FIG. 5a shows a NSOM probe with embedded metal nanowire optical antenna.

FIG. 5b shows another NSOM probe modified with an embedded metal nanowire optical antenna and coated with a dielectric and second metal, the fundamental basis of an individual nanocoax optrode.

FIG. 5c shows an expanded view of middle image, showing constituent components.

FIG. 5d depicts alternate route to coaxial NSOM optrode, prepared by pulling a glass pipette into a sharp point with an etched W wire inside (SEM inset), and then coating with insulator (Al₂O₃) and shield (Au).

FIG. 6a shows a SEM of an individual nanocoax.

FIG. 6b shows an SEM of an NCOA of vertically oriented nanocoaxial structures.

FIG. 6c-f displays optical micrographs of infrared (false color), red, green and blue (all true color) polarized light propagated along and out of a nanocoax. Dashed lines indicate boundaries of coax annulus.

FIG. 6g shows a 3D contour plot of a 20×20 μm² array.

FIG. 7 shows extracellular recording of a spontaneous action potential from a neuron (Retzius cell) in a ganglion sac extracted from a Hirudo Medicinalis (leech) nerve cord as measured by a nanocoax sub-array in direct contact.

FIG. 8a shows infrared differential interference contrast image (IR-DIC) of pyramidal neuron (yellow arrow) in a whole cell patch configuration (black arrow).

FIG. 8b shows a voltage trace shows depolarization to 50 pA current injection (black trace) or 473 nm blue light administered through the NSOM probe at increasing power.

FIG. 8c depicts that, after wash out of tetrodotoxin and kynurenic acid, 5 ms light “injections” evoked synaptic response akin to glutamate mediated AMPA currents.

FIG. 9a depicts a NIL nanopillar fabrication process: SEM images of a master array of 200 nm diameter, 2 μm tall Si nanopillars, an imprinted negative stamp, and an SU-8 polymer replica of the master.

FIG. 9b shows a photograph of a 10×20 mm² chip with 9 subarrays of nanocoaxes wired as differential addressable and 8 in a network configuration. Each measures 500 μm on a side.

FIG. 9c shows an optical micrograph of back-illuminated, flexible nanocoax array (1.5 μm pitch), showing light has propagated through the annulus of each coax.

FIG. 9d shows an SEM image of an optically transmitting nanocoax-based array (NCOA) prepared with protruding cores is shown in, which may facilitate cell immobilization and potential intracellular recording.

FIG. 10 shows a schematic of LED-coupled NCOA subarrays, spatially separated.

### DESCRIPTION OF THE INVENTION

The invention provides novel devices and methods that enable ultrahigh spatial and temporal resolution interfaces, which allow access and intervention to local (intra- and proximate extra-neuronal) neuroelectronic and neurotransmitter molecular signatures associated with aberrant cell function and cell death leading to neurodegenerative diseases.

Scalable devices based on a unique nanocoaxial electrode array of the invention offer neural recording and control at unprecedented levels of precision. The optrode array disclosed herein exceeds the spatial resolution of existing MEAs while uniquely incorporating simultaneous light delivery and electronic recording in a single device element. Nanoscale spatial resolution is immensely valuable offering the ability to generate spatially resolved images of the dielectric properties of individual biological specimens, to monitor the response in time, space and electromagnetic frequency to an electrical disturbance introduced at a specific site in a specimen, or to other external perturbations such as light, pressure, sound, temperature, or the introduction of chemicals/drugs.

Traditionally, the study of electrogenic cells has been through the use of patch clamp techniques and devices capable of addressing multiple neurons, such as microelectrode arrays, nanowire electrode arrays, field effect transistor arrays, nanopillars arrays and engulfing microprotrusions. (Spira, et al. 2013 Nature Nanotech. 8:83-94; Hierlemann, et al. 2011 Proc. IEEE 99:252-284; Robinson, et al. 2012 Nature Nanotech. 7:180-184; Duan, et al. 2012 Nature Nanotech. 7:174-179. Voelker, et al. 2005 Small 1:206-210; Xie, et al. 2012 Nature Nanotech. 7:185-190; Hai, et al. 2009 J. R. Soc. Interface 6:1153-1165.) Extracellular recording often suffers from reduced coupling to the cell membrane, such that typical electrical signals associated with action potentials can be a fraction of the full trans-membrane potential difference, potentially comparable to interfering signals and noise transients. Furthermore, these tools do not provide nanoscale (sub-micrometer) resolution or precision. Instead, one has to choose between electrical precision (patch clamp) or spatial resolution (microelectrode arrays or MEA) but not both. Importantly, the spatial resolution of commercially available MEAs is limited relative to the dimensions of neurons.

Advances in genetically encoded light-sensitive molecules have allowed the membrane potentials of neurons to be controlled in a temporally precise fashion by brief pulses of light. The so-called “optogenetic” strategy offers advantages over previous pharmacological and electrophysiological methods, namely cellular and temporal specificity. (Fenno, et al. 2011 Ann. Rev. Neurosci. 34:389-412.) The principal component of an optogenetics experiment is an “optrode”, a hybrid optical stimulator for light delivery and electrode for recording neural activity. (Gradinaru, et al. 2009 Science 324, 354-359.)

Existing optrodes are, however, rudimentary devices with very limited spatial resolution and separate channels required for optical and electrical functions. Current devices are restricted to recording from only a few electrode channels and it is challenging to target individual cells. A number of variations on the optrode have been reported, but high spatial resolution, multiple input/output channels and closed circuit/loop integrated control remain unmet challenges in optrode development. (Zorzos, et al. 2012 Opt. Lett. 37:4841-4843; Deisseroth 2011 Nat. Methods 8:26-29.)

Devices of the invention, as neurophysiological tools, address the following requirements of a preferred architecture: (1) high scalability—the ability to record and stimulate large numbers (tens to millions) of individual neurons simultaneously without compromising cell viability, (2) robust electronic coupling to neurons over extended periods of time (hours to months), and (3) ability to detect synaptic events and ion channel/membrane physiology.

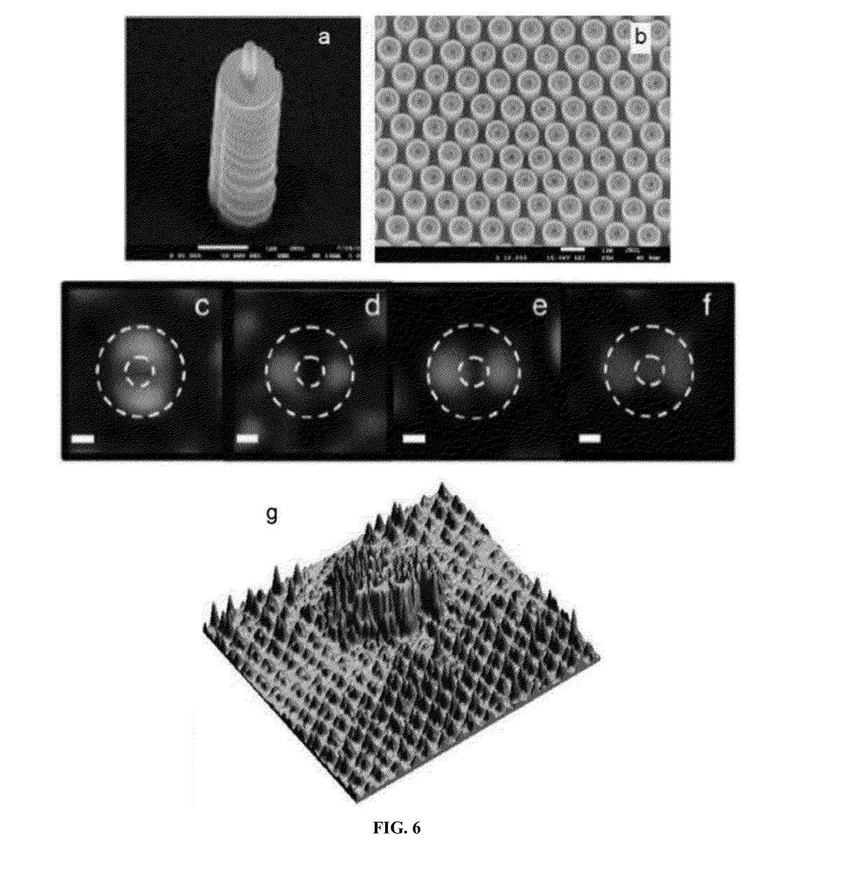

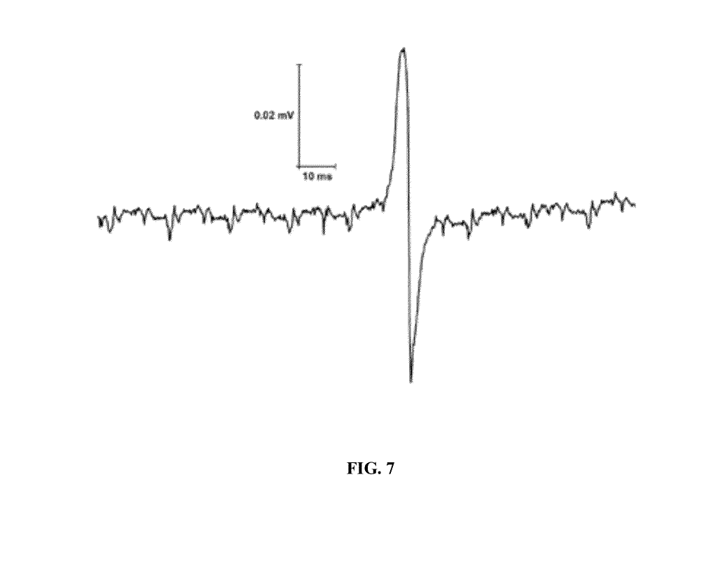

In certain aspects of the invention, a shielded electrode array architecture is employed as a neuroelectronic interface with an ultrahigh spatial resolution. The shielded electrode array architecture is comprised of a networked array of shielded nanoscale coaxial electrodes. An example of a nanocoax array is shown in the embodiment of FIG. 6b. The array of some embodiments is configured for use in extracellular recording of neuronal activity. In other embodiments, the array is configured for nanoscale intracellular interrogation.

Embodiments of the invention include a label-free, fusion multiplex device that spatiotemporally correlates neuroelectronic activity at thousands of discrete sites, and combines electrical detection of neuronal activity with that of neurotransmitter release.

Any potential issues with crosstalk between closely-spaced electrodes is addressed by having nano-electrodes with local electrical/electromagnetic shielding. The local shield serves to confine electric potential variations to the vicinity of each electrode, reducing if not eliminating inter-electrode crosstalk, and restoring spatial resolution to that of the electrode pixelation level, which can be submicroscale.

In addition to utilization as an extracellular current/voltage-sensing device of neuroelectronic activity (action potentials and their propagation along and between neurons), embodiments of the array structure are configured to monitor neurotransmitter release with the same high spatial acuity, and with high sensitivity and selectivity. This array configuration can be used, for example, as an ultrasensitive chemical and molecular sensor. This includes antibody (Ab) approaches, (e.g. anti-dopamine, anti-glutamate, anti-histamine, anti-serotonin), and molecular imprint polymer (MIP) approaches. Both Ab and MIP also afford the detection of extracellularly accumulated macromolecules, such as peptides and proteins (e.g., abnormally folded amyloid beta and amyloid tau proteins found in Alzheimer's disease).

Embodiments of the array structure may be used for electrical impedance spectroscopy (EIS)-based, spatial mapping of the topographical substructure of individual biological entities such as cells, neurons, proteins, and tumors, in terms of their dynamic electrical impedance, by the use of proximity sensing with terminated coaxial cables. Array structure embodiments may further enable monitoring of temporal and spectroscopic characteristics on sub-cellular spatial levels, via the time dependence of the electrical impedance of nanoscale regions of a cell, as well as of numerous nanoscale regions simultaneously, for example, during cell growth.

An array embodiment entails the use of a nanocoax electrode for ex situ (in vitro or in silico) extracellular interrogation of synaptic activity when a neuron or other electrogenic cell is placed in close proximity to the electrode, such as less than about 100 nm or less than about 10 nm away/above the top of the electrode. Also, an array embodiment entails the use of a nanocoax electrode for in situ (in vivo) extracellular interrogation of synaptic activity when a neuron or other electrogenic cell is placed in close proximity to the electrode, such as less than about 100 nm or less than about 10 nm away/above the top of the electrode. These embodiments further enable the monitoring of the response of specific, user-selected regions (with nanoscale precision) of a cell to electrical and electromagnetic stimuli introduced at user-selected regions (with nanoscale precision) of the same or a neighboring cell or other biological entity. Such a response may include the direction, promotion, or inhibition of cell growth.

Embodiments of the invention further enable nanoscale-resolution measurement and monitoring of cell motility (movement) on a substrate in response to external influences (e.g., light, heat, electrical impulse, pressure, or introduction of chemicals or drugs). Electrical stimulation (AC or DC) can be introduced at a specific site or sites at the surface of a cell/entity with nanoscale resolution, and the subsequent monitoring of the effect(s) of such stimulation at a different site or sites as functions of time, distance and frequency. These attributes can facilitate the identification of correlations between dielectric activity at locally identified sites in a specimen and specimen motility, viability, disease state, and drug response, for in vivo as well as in vitro applications.

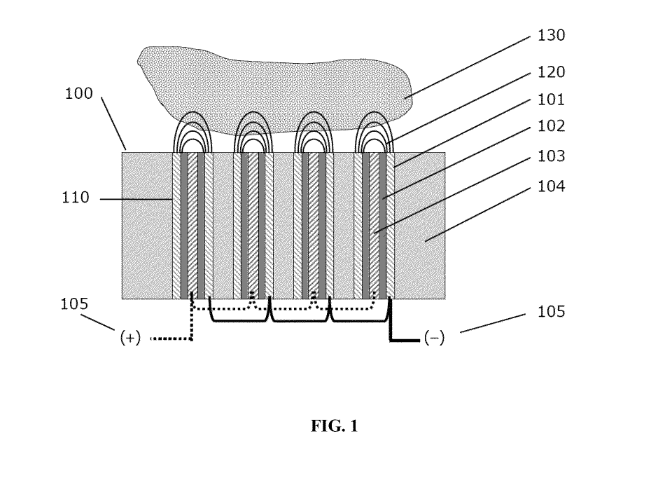

In the exemplary embodiment of FIG. 1, four vertically oriented nanocoaxes are in an array 100. The nanocoaxes are embedded in a mechanical stabilizing medium 104. When an alternative current electric potential is applied to electrodes 105 connected to the inner 103 and outer 101 conductors (separated by a dielectric material 102 in the annulus) of each open-ended nanoscale coaxial line 110, a stray electric field 120 penetrates the vicinity of each coax end. When a biological specimen (e.g., cell) 130 is situated on or near the surface of the array 100, electric field lines 120 will penetrate the specimen in localized volumes. The dielectic properties of the specimen, characterized by the dielectic permittivity ε=ε(ω,t,x,y,z), which can be a function of electrical excitation frequency ω/2π, time t and spatial dimensions x, y and z (as well as other intrinsic and extrinsic parameters) will cause a change in the electrical impedance of each coax in its vicinity. This change is detected by measuring the complex impedance (resistance, capacitive reactance and inductive reactance) of each coax. As such, spatial, temporal and frequency spectral variations in dielectric impedance can be mapped. In another embodiment, changes in the local field potentials due to synaptic activity at the locations of the nanocoaxes can be detected as changes in the potential (voltage) at each coax.

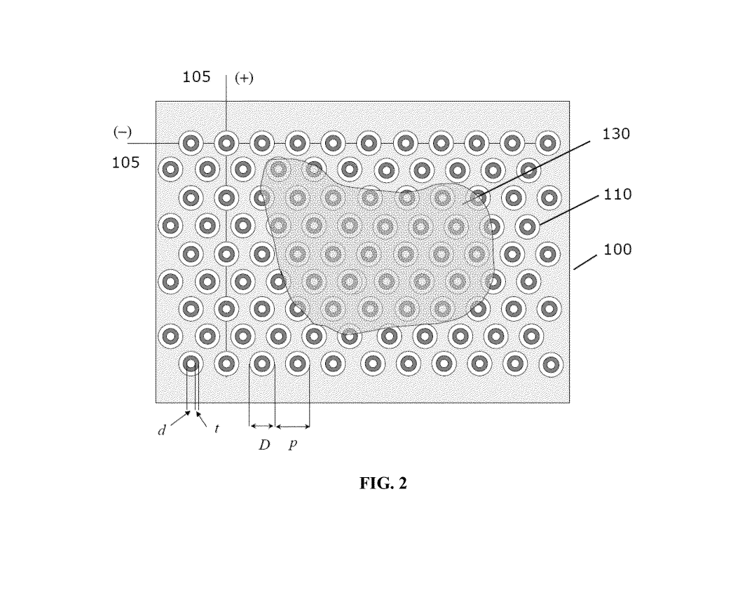

In another exemplary embodiment shown in FIG. 2, an array 100 is comprised of vertically oriented, open-ended nanocoaxes 110. Each coax is comprised of a highly conducting or metallic inner conductor of diameter d, radially coated with an insulating dielectric medium of thickness t, and an outer highly conducting or metallic, cylindrical layer with inner diameter of about d+2t and outer diameter D (e.g., of thickness D/2−d/2−t). The inner conductors 105 of all coaxes are connected together, and the outer conductors 106 of all coaxes are connected together.

In another embodiment, the nanocoax electrode array for in situ (in vivo) is employed in extracellular interrogation of synaptic activity when a neuron or other electrogenic cell is placed in close proximity to the electrode, such as less than about 100 nm or less than about 10 nm away/above the top of the electrodes. In this embodiment, nanocoax electrodes are monitored, simultaneously or sequentially, such that the propagation, in space and time, of an action potential can be monitored, with spatial resolution determined by the pitch or lateral spacing of the electrodes. Such spacing can be less than about 1 μm, or less than about 10 μm, or less than about 100 μm, with typical values between about 0.3 μm and 3 μm. The number of coaxes varies in different embodiments. For example, the number of coaxes can be 2, 10, 100, 1000, or more.

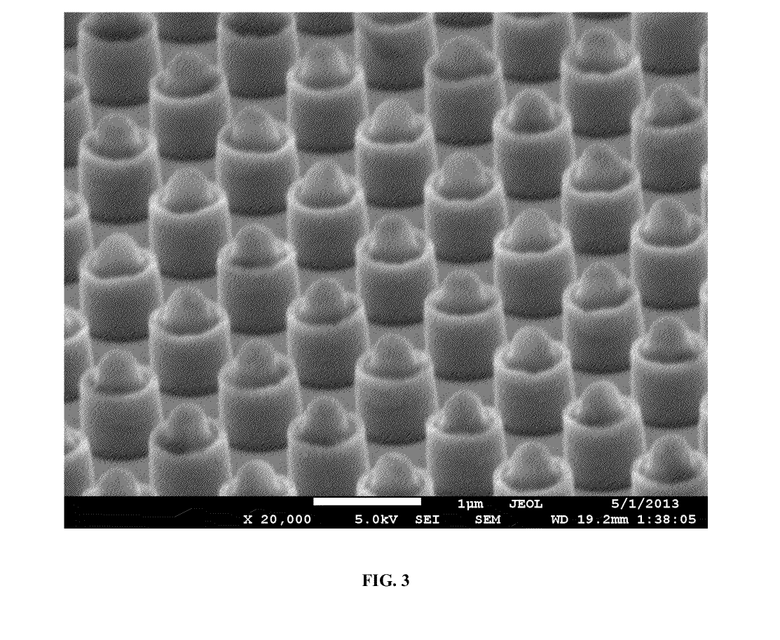

In certain embodiments of the nanocoax electrode, the length of the inner conductor is longer than that of the outer conductor, such that the inner conductor protrudes vertically out of the coax structure. In FIG. 6a, an individual nanocoax has a protruded inner conductor; and in FIG. 3, the nanocoax electrodes in the array have protruded inner conductors. With such a protruded structure, the nanocoax electrode can facilitate intracellular, in addition to extracellular, recording of synaptic activity. That is, the protruded inner conductor can be configured to pierce the cell membrane and thereby extend inside the cell, leaving the outer coax conductor outside the cell, in the intercellular region. When such an electrode pierces a cell membrane and gains access to the intracellular region, an electrical measurement (e.g., potential difference, or voltage) is made relative to the proximate second electrode (the coax outer conductor).

With the protruded conductor architecture, each of the above extracellular embodiments is available as an intracellular embodiment. Each of the intracellular and extracellular structures can be operated in a number of passive and active recording modalities, all as functions of space (position on an array) and time. These include passive and active voltage and current sensing/recording, and active electrical impedance spectroscopy. In addition to all of the above sensing/recording modalities, one or more shielded nanocoaxes (protruded version or not) can function as a stimulation electrode, such that a voltage or current pulse or signal can be sent from a nanocoax to a proximate cell to stimulate the initiation of an action potential.

An example of sensing by a nanocoax array of leach neural activity is shown in the embodiment of FIG. 7. In certain embodiments, the nanocoax is employed as an input to a voltmeter or current meter, and is used to directly sense transient changes in the local electric field potential (extracellular recording) or currents within as much as several diameters of the top surface of the nanocoax. In this voltage/current-sensing mode, the nanocoax may bring the signals out to extra-chip electronics, or amplification elements such as transistors can be integrated on the nanocoax chip.

Embodiments of this mode are characterized through the DC or AC biasing of the nanocoax core relative to the shield. The equipotential lines from the fringing capacitance of the coax embodiment extend above the terminated ends out as much as several coax diameters, and into the biological media solution. Distortions in these field lines can show up as changes in the coax impedance or voltage/charge changes on the biased coax. This can be used for high resolution spatial proximity sensing, or to induce an action potential through highly localized stimulation of a coupled neuron.

In certain embodiments, the nanocoax array can be used to record chemical signatures of synaptic activity from neurons and other electrogenic cells, by virtue of sensing neurotransmitter release. Embodiments of the nanocoax array can also be useful as ultrasensitive chemical and molecular sensors. This technique can be used to configure a nanocoax, or array of nanocoaxes, to monitor neurotransmitter release with high spatial acuity, and with high sensitivity and selectivity. Target neurotransmitters that possess antibodies can be detected using antigen-antibody binding. The antigens (e.g., anti-dopamine, glutamate, histamine, serotonin) bound by biochemical surface functionalization techniques to either the outer surface of the nanocoax inner conductor, or the inner surface of the outer nanocoax conductor. Other embodiments may be used with molecular imprint polymer approaches. Furthermore, embodiments of the device can include both electrical detection of neuronal activity and neurotransmitter release.

A key unique aspect of the invention is the coaxial electrode. In exemplary embodiments, the nanocoaxial electrode has a conductive inner core surrounded by a dielectric shield encased by a conductive outer layer. Such a construct has three concentric layers: conductive inner core, dielectric shield, and enclosing conductor/shield. For instance, one can fabricate coaxial wires on the nanoscale such that the diameter of the outer layer is <1 μm (preferably less than 500 nm) and in arrays with a spatial pitch of 10 μm or less (preferably less than 1 μm).

Importantly, each coaxial electrode device can serve as both a nanoelectrode and a nanooptical element. When combined with optogenetic technology, the dual optoelectronic functions of the nanocoax architecture allow for novel and creative solutions that are scalable from subcellular, to cellular, to circuit and, finally, to volumetric neural applications.

While optogenetics has provided powerful tools for the control of membrane potentials in mammalian neurons with light, extensive limitations remain with existing technologies. The nanocoax architecture of the invention addresses these limitations by offering a spatial resolution that far exceeds what is required to control and record from an individual neuron.

First, the inner and outer conductors of the nanocoaxial electrode serve as conventional electrodes for extracellular recordings, or the inner core can pierce a cell for intracellular records. Second, the dielectric material can be optically transmitting and can be coupled to a light source to locally illuminate the tissue. Third, the coax can be prepared with an electroluminescent dielectric medium (chromophores or quantum dots), enabling it to function as a light emitter for spatially and spectrally discrete output (e.g., blue vs. infrared light at specific X, Y coordinates).

Nanoscale coaxial electrodes can be fabricated with properties that, when combined with optogenetics, are ideal for monitoring and controlling neural activity. By constructing arrays of nanocoaxial electrodes, with variable and designed lengths, light can be discretely administered to a region of interest, in precise 3-D coordinates while simultaneously monitoring the electrophysiological response to that light in the surrounding volume of tissue. A device of multiplexed, variable depth arrays can be deployed in light sensitive brain tissues, for example, for implantable and volumetric neuronal control.

In a nanocoax electrode having a conductive inner core surrounded by a dielectric annulus encased by a conductive outer layer, the inner core and outer shield are used as recording and reference electrodes for highly spatially-resolved intracellular and extracellular recordings at a scale down to about 10 μm² or smaller pixel. Importantly, the dielectric layer can be fabricated as a nanoscale optical light guide, light emitting diode, or electroluminescent, which allows for spatially discrete illumination at the coaxial wire tip and onto proximate optogenetic neurons. This nanocoax architecture provides an “optrode” interface for simultaneous optical and electrical manipulation and recording; the critical element of optogenetics.

Individual nanocoaxial optrodes or addressable, two-dimensional nanocoaxial optrode arrays (NCOAs) can be fabricated. The NCOA combines the optical waveguiding properties of a nanoscale coaxial cable, with that structure's intrinsic electrical sensing capability, in a hybrid optoelectronic device that can deliver light simultaneous to highly spatially-resolved electrical recording. (Rybczynski, et al. 2007 Appl. Phys. Lett. 90:021104; U.S. Pat. Nos. 7,589,880; 7,623,746; 7,634,162; 7,649,665; 7,754,964; 7,943,847; 8,431,816 and 8,588,920; Kempa, et al. 2008 Appl. Phys. Lett. 92:043114.) The NCOA array interface will thus allow for multiplexing and optogenetic interrogation on scales that have not previously been achievable. Individual wires can be clustered, addressed and multiplexed according to the target, such as a single neuron's action potential might be recorded by dozens of electrodes. In the case of neural transmission, the smallest electrochemical element might be a single dendritic spine (about 1 μm²), which is much larger than the core electrode of a single nanocoax. Furthermore, in the optogenetic preparation, individual pixels can function as either light emitters or electrodes calibrated to the specific application. These features separate the nanocoax from the single mode, ‘nanopillar’ architectures that only offer electronic interrogation and, lacking the local shield of a coax, are unable to reach the pixel scale of the nanocoax.

The nanocoaxial electrode can be fabricated in 3D optrode arrays that can be implanted to neural tissue for in vivo optoelectronic control. The NCOA is highly configurable such that individual optrodes can be tuned for delivery of light at specific wavelengths based on local electrical measurements. The NCOA enables one to: (1) observe neuronal circuit activity with high spatial (approaching micron scale) resolution; (2) deliver light with the same spatial resolution; and (3) dynamically and spatially control the delivery of light to tissue based on field recordings in extremely close proximity to each other, thus enabling closed-loop, optoelectronic feedback toward in situ control over neuroelectronic function.

Importantly, a unique aspect of the coaxial nature of the proposed NCOA device is its integrated local electrical shielding for each electrode. This shielding serves to eliminate inter-electrode cross-talk and ambient noise that otherwise would increase as unshielded sensing electrodes are brought into close proximity (e.g., at high density). The neuroelectronic interface based on the nanocoaxial arrays extends the spatial precision to the nanoscale and incorporate the enormous advantages of optogenetics into a highly advanced optrode array.

The nanoscale electrode array disclosed herein is scalable to fine resolutions. For example, the improved resolution can permit the observation of electrical fields from cellular compartments, e.g. a dendritic spine head at about 1 μm², a significant improvement over existing technologies.

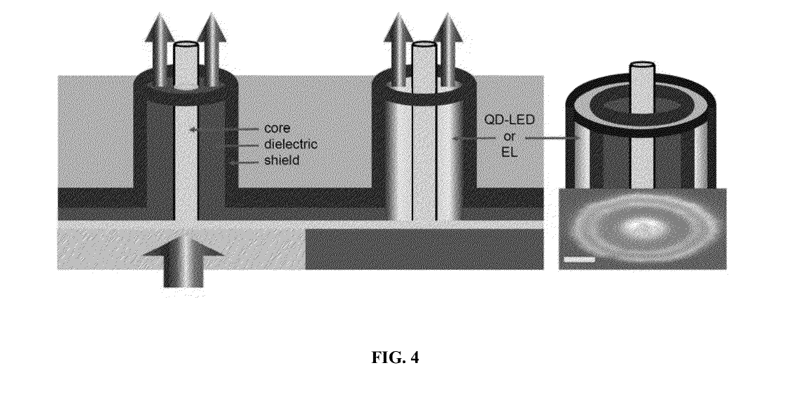

FIG. 4 depicts schematics of the basic structure in optoelectronic implementations, as a nanoscale optrode device. A light-transmitting (left, FIG. 4a) or light-generating (center, FIG. 4b and right, FIG. 4c) coax annulus facilitates highly localized optical stimulation, while electrically conducting inner (core) and outer (shield) coax electrodes enable localized neuroelectronic detection. The scheme at right (FIG. 4c) extends the coax concept to a triaxial architecture, wherein the light-generating, e.g., a quantum dot-based light-emitting diode, QD-LED or electroluminescent (EL), medium resides between the outer two cylindrical electrodes. This construct leaves the central core as a neuroelectronic probe electrically isolated from and unaffected by the optical function. Also shown is an SEM image (FIG. 4d, scale bar: 200 nm) of an actual triaxial device.

To improve resolution of neural circuits, the nanocoax's optical and electrical capabilities are augmented by reducing the inter-electrode spacing or pitch of electrodes while simultaneously advancing back end electronics for massively multiplexed data acquisition. Control of optogenetic systems is improved by allowing spatially (in 3D) precise, electrode feedback regulated optical interrogation of neural tissue.

### Fabrication of Individual Nanocoax Optrode

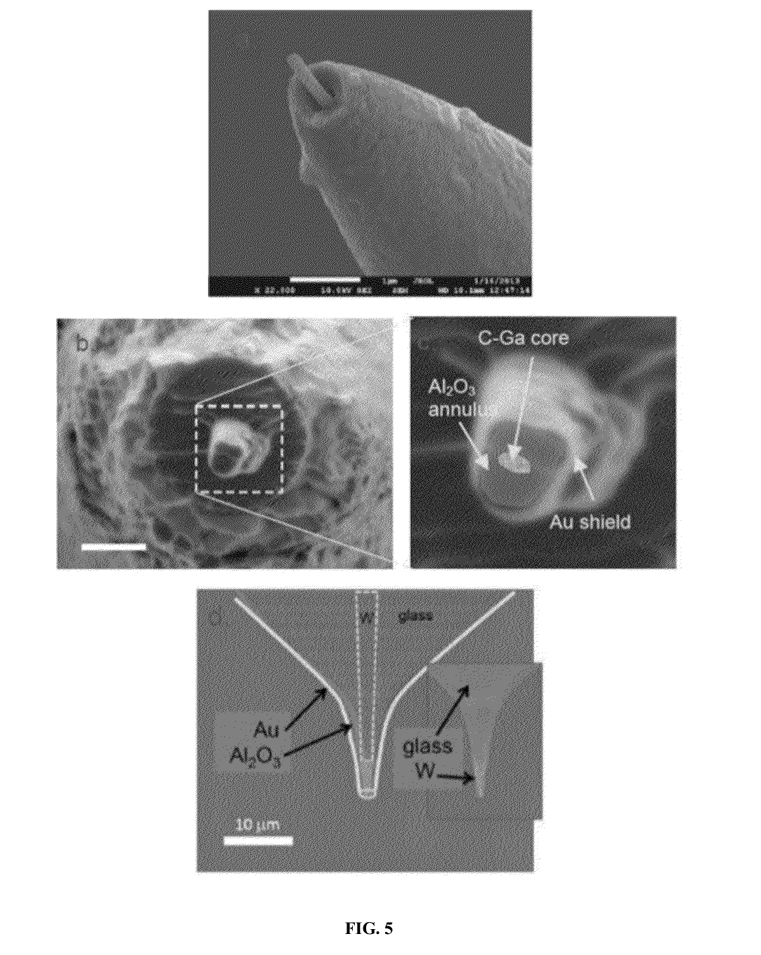

Depicted in FIG. 5, with a scale bar of 1 μm, is a probe consisting of a metal-coated, tapered optical fiber was modified for enhanced light delivery by embedding a nanowire antenna within the dielectric (glass) core, and for electrical sensing by coating the metal with a dielectric insulator and a second metal. To this end, as shown in FIG. 5, a 150 nm-diameter W—Ga nanowire was grown by focused ion beam (FIB) deposition into the apex of a near-field scanning optical microscope (NSOM) probe, a FIB-deposited 90 nm-diameter C—Ga wire subsequently coated by a dielectric (Al₂O₃) and a metal (Au), and a pulled glass pipette that had an etched W wire placed inside prior to pulling.

In the construction in FIG. 5b and FIG. 5c, the end of the device was exposed by FIB-milling, revealing the metal-dielectric-metal that serves as the optical waveguide. Additional dielectric and metal coatings are placed to form the integrated electrical sensor (e.g., a triaxial device). FIG. 5a shows a NSOM probe with embedded metal nanowire optical antenna. FIG. 5b, with a scale bar: 500 nm, shows another NSOM probe modified with an embedded metal nanowire optical antenna and coated with a dielectric and second metal, the fundamental basis of an individual nanocoax optrode. FIG. 5c shows an expanded view of middle image, showing constituent components. FIG. 5d depicts alternate route to coaxial NSOM optrode, prepared by pulling a glass pipette into a sharp point with an etched W wire inside (SEM inset), and then coating with insulator (Al₂O₃) and shield (Au).

### Fabrication of Nanocoaxial Optrode Arrays

FIG. 6a shows a SEM (with a scale bar of 1 μm) of an individual nanocoax with about 100 nm thick Al₂O₃ annulus (W core, Cr shield). FIG. 6b shows an SEM with a scale bar of 1 μm that depicts an NCOA of vertically oriented nanocoaxial structures, with coax annuli that are capable of propagating visible light, and with inner and outer coax conductors that can serve as local electric potential probe pairs. An experimental device was constructed using 2 cm² area arrays of Si nanopillars (0.2 μm diameter, 2 μm height at 1.3 μm pitch) on Si substrates. Nanoimprint lithography can be to prepare polymer nanopillar arrays with the same dimensions. Coaxial electrodes were then prepared by sequential metal, dielectric and metal coatings onto the nanopillars. In order to prepare this structure for neuroelectronic recording and stimulation, the inner electrode must be exposed. This was achieved by polishing the array, thereby “decapitating” the structures and leaving behind the open-ended nanoscale coaxial electrodes shown in FIG. 6b. In order to facilitate this polishing, a polymer film was spin-coated over the array and hardened, mechanically stabilizing the structure.

### Optics of Nanocoaxial Optrode Arrays

FIG. 6c-6f shows images that demonstrate subwavelength optical propagation in the coax. Polarized lasers were coupled into the bottom of a nanocoax, with the light emanating from the top end recorded by high-resolution optical microscopy. (Merlo, et al. “Experimental observation of the TM propagated modes in nanocoax structures (submitted for publication).) FIG. 6c-6f displays optical micrographs (a scale bar of 200 nm) of infrared (false color), red, green and blue (all true color) polarized light propagated along and out of a nanocoax. Dashed lines indicate boundaries of coax annulus. FIG. 6g shows a 3D contour plot of a 20×20 μm² array, showing spatially specific light delivered to a small region (through about 20 nanocoaxes). As shown are integrated density (red=high, violet=low) by x and y spatial directions. Photocurrents elicited in slices on top of this array are constrained to the illuminated region. Importantly, light delivered to a small area of a larger version of the array can be spatially limited, as shown in the subarray contour plot (FIG. 6g).

### The Nanocoax as an Electrode

An optrode array can be constructed for advanced neurological sensing and control. In addition, chemical and electrochemical sensing can be achieved. For the former, sub-part-per-billion sensitivity was achieved, while the latter exhibited about 100×higher sensitivity than a conventional electrochemical control cell. (Zhao, et al. 2012 ACS Nano 6:3171-3178; Rizal, et al. 2013 Anal. Chem. 85:10040-10044.)

In one study, a 0.25 mm² subarray containing about 10,000 nanocoaxes wired in parallel was used. Electrical transients were recorded using neurons dissected from a live specimen of the medicinal leech, Hirudo Medicinalis. To test the efficacy of the device, Retzious cells from the leach abdominal ganglion were dissected, placed over the nanocoax subarray, and local field potentials reflecting spontaneous action potentials were sensed. (Muller, et al., Eds., “Neurobiology of the Leech”, Cold Spring Harbor Laboratories, Cold Spring Harbor, N.Y., 1982.)

FIG. 7 shows the observed signal, a biphasic waveform indicitative of extracellular recordings, consistent with results from leech neurons. (Jenkner, et al. 1997 Phys. Rev. Lett. 79:4705-4708.) Measured by a nanocoax sub-array in direct contact, extracellular recording was achieved of a spontaneous action potential from a neuron (Retzius cell) in a ganglion sac extracted from a Hirudo Medicinalis (leech) nerve cord. It was established that the impedance of the recording electrode to the shield ground was about 10 kΩ, which is in the range employed for conventional extracellular recording. It was also demonstrated that neurons can be positioned or cultured directly on top of the device surface, with no apparent detrimental effect to the cell membrane and recordings made for several hours, which is about the lifetime of the particular neurons (leech) employed.

Thus, in one aspect, the invention generally relates to a nanocoaxial electrode. The nanocoaxial electrode includes: a conductive inner core; a coaxial dielectric layer surrounding the conductive inner core; and a coaxial conductive outer layer encasing the dielectric layer. The coaxial dielectric layer is adapted to cover the conductive core, and the diameter of the coaxial conductive outer layer is less than about 1 μm.

In certain embodiments of the nanocoaxial electrode, the conductive inner core and conductive outer layer are adapted to serve as recording and reference electrodes. In certain embodiments, the nanocoaxial electrode further includes an embedded metal nanowire optical antenna.

In certain embodiments of the nanocoaxial electrode, the inner core is constructed as a nonconducting “innermost” core coated with a conductor.

In certain embodiments, the coaxial dielectric layer is adapted to serve as an optical light guide. In certain embodiments, the coaxial dielectric layer is adapted to serve as a light emitting diode. In certain embodiments, the coaxial dielectric layer is adapted to serve as an electroluminescent component providing spatially discrete illumination.

The conductive inner core can be made from any suitable conductive material, for example, one or more selected from Ag, Au, C—Ga, W—Ga, Ni, Cr, Ti, Al and IrOx. The dielectric layer can be made from any suitable dielectric material, for example, one or more selected from Al₂O₃, SiO₂ and SU-8. The conductive outer layer can be made from any suitable conductive material, for example, one or more selected from Ag, Au, Cr, Ti, Al, Pt, C, W and Ni.

The nanocoaxial electrode usually has a dimension wherein the diameter of the coaxial conductive outer layer is less than about 2 μm (e.g., less than about 1 μm, less than about 500 nm, less than about 400 nm, less than about 300 nm, less than about 200 nm, between about 100 nm to about 1 μm, between about 100 nm to about 500 nm, between about 200 nm to about 1 μm, between about 200 nm to about 500 nm, between about 150 nm to about 500 nm, between about 150 nm to about 300 nm).

In another aspect, the invention generally relates to a nanocoaxial optrode array. The nanocoaxial optrode array includes: a plurality of inter-connected nanocoaxial electrodes arranged in an array; a light delivery component; and an electronic recording component. The nanocoaxial electrode includes a conductive inner core; a coaxial dielectric layer surrounding the conductive inner core; and a coaxial conductive outer layer encasing the dielectric layer, and wherein the diameter of the coaxial conductive outer layer is less than about 500 nm.

In certain embodiments, the nanocoaxial optrode array is coupled to a light emitting diode (LED) array.

In certain embodiments of the nanocoaxial optrode array, the nanocoaxial electrodes have a protruding inner conductive core. In certain embodiments, the coaxial dielectric layer of nanocoaxial electrodes is adapted to serve as an optical light guide. In certain embodiments, the coaxial dielectric layer of nanocoaxial electrodes is adapted to serve as a light emitting diode. In certain embodiments, the coaxial dielectric layer of nanocoaxial electrodes is adapted to serve as an electroluminescent component providing spatially discrete illumination.

In certain embodiments of the nanocoaxial optrode array, the nanocoaxial electrode further includes an embedded metal nanowire optical antenna.

The invention also relates to embodiments of fabrication of the nanocoaxial electrode, the nanocoaxial optrode array, and devices having them as components.

In yet another aspect, the invention generally relates to a method for detecting extracellular neuronal activity. The method includes: providing a neuroelectronic probe comprising one or more nanocoaxial optrode arrays; contacting the neuroelectronic probe with a tissue sample; and manipulating the neuroelectronic probe to detect extracellular neuronal activity of the tissue sample.

In certain embodiments, the method further includes recording extracellular neuronal activity of the tissue sample. In certain embodiments, the method is performed in vitro. In certain embodiments, the method is performed in vivo.

### EXAMPLES

### Individual Nanocoax Optrodes for Optogenetic Electrophysiology

Previously, the capabilities of the nanocoax as an electrical sensor and as a subwavelength waveguide for visible light were demonstrated. (Rybczynski, et al. 2007 Appl. Phys. Lett. 90:021104; Zhao, et al. 2012 ACS Nano 6:3171-3178; Rizal, et al. 2013 Anal. Chem. 85: 10040-10044; Rizal, et al. 2013 NATO Sci. for Peace & Security Series B: Physics and Biophysics XIX:359-372.) These functions are adopted by preparing individual nanocoaxes as optrodes for simultaneous optical stimulation and electrical recording compatible with optogenetic neurobiological preparations. Their use for light delivery is validated by characterizing their optical throughput properties, for example, using Nanonics MultiView 4000 NSOM system. Fiber optic coupling can be used to deliver white, laser, or LED light to the nanocoaxial optrodes. Relevant to optogenetic application, the efficiency of optical transmission at blue (473 nm) or green (561 nm) wavelengths, and the power emitted at the optrode tip are measured in the far field by photodetectors positioned opposite the optrode, and by NSOM.

The range of achievable output power is then determined with the preferred optical intensity equivalent to 10 mW/mm² at the working end without generating deleterious heat. Optical throughput is greatly facilitated by the use of the nanowire antenna positioned in the core of the tapered fiber. As there is a trade-off between throughput and spatial resolution, controlled by adjusting the thickness of the insulating layer around the nanowire core, a series of probes and materials are prepared and tested.

Examples of fabricated probes include nanocoax cores from Ag, Au, C—Ga, W—Ga, Ni, Cr, Ti, and Al metals, with Ag, Au, Cr, Ti and Al variously used for the shield material. For example, in a Ti:Au system Ti is employed as the core and/or shield. Also, iridium oxide (IrOx) can be utilized as it is well-known as biocompatible as a neuroelectrode. (Cogan 2008 Annu. Rev. Biomed. Eng. 10:275-309.) Highly conformal IrOx films can be deposited by atomic layer deposition (ALD). (Hämäläinen, et al. 2011 J. Mater. Chem. 21:16488-16493.) Similarly, a number of materials are available for the dielectric in the coax annulus, with Al₂O₃, SiO₂ and various polymers employed to date.

Another embodiment, an alternative to waveguiding externally generated light, is intrinsic light generation. This can be achieved either by integrating into the coax annulus a radial p-n junction, a film containing light-emitting quantum dots, or an electroluminescent (EL) medium. The NSOM probes with light-generating capability can be fabricated and tested for power output. Electroluminescence creates photons by radiative recombination of electrons and holes, usually in a semiconductor, or at the interface between two semiconductors as in a light emitting diode (LED). By replacing the annular dielectric on a nanocoax with an electroluminescent medium, one can make a light emitting nanocoax (LENC).

In an alternative approach, a conventional nanocoax can be fabricated and is overcoated with the electroluminescent medium and another metallic layer, thus creating a triaxial structure, as depicted in FIG. 4. Such three-contact structure allows one to ground the middle metal layer and bias the outer contact (relative to ground) to stimulate the electroluminescent medium, while monitoring the inner most contact (relative to ground) to sense the neural signals.

One method to create LENCs uses the standard thin film fabrication techniques as are used for conventional nanocoaxes, but to replace the Al₂O₃ dielectric with electroluminescent oxides or sulfide thin films, such as sputtered ZnO—Bi₂O₂ bilayers, sputtered ZnS:Mn (zinc sulfide doped with Mn) films or by atomic layer deposition (ALD) of ZnS, SrS, and BaS thin films. (Nakajima, et al. 1991 Proceedings of the 3rd International Conference on Properties and Applications of Dielectric Materials, Tokyo Japan, pp. 181-184; Gupta, et al. 1997 Thin Solid Films 299:33-37; Ihanus, et al. 2005 J. App. Phys. 98:113526; Ihanus, et al. 2003 J. App. Phys. 94:3862-3868; Ihanus, et al. 2002 Chem. Mater. 14:1937-1944.) Most of these materials emit in the green-blue region of the visible spectrum, and can be tuned from about 450 nm to 550 nm peak emission wavelength. These materials have electroluminescent decay times appropriate for pulsing, if need be (e.g., on 100 μs times scales). A typical layer structure for a ZnS based LENC would be to replace the Al₂O₃ dielectric with a ZnS (doped with Mn or Cu)—Al₂O₃ or ZnS (doped with Mn or Cu)—SiO₂ sandwich, with the existing metal core and shield used as the EL drive contacts.

An alternative architecture is to make a conventional nanocoax that is then coated with the EL layers on its outside, with a third outer metal layer. The nanocoax core and shield is used to read the neurophysiology signals, and a bias between the shield and outermost metal would excite the EL materials.

Another method to incorporate an electroluminescent medium into a nanocoax or nanotriax utilizes quantum dots as the light-emitting medium. (Dabbousi, et al. 1995 Appl. Phys. Lett. 66:1316-1318.) An advantage of quantum dots is their wide emission tunability, allowing the output wavelength to be tailored for specific excitation needs. Quantum dot systems can be fabricated as tunable over the entire visible spectrum using commercially available materials. (Anikeeva, et al. 2009 Nano Lett. 9:2532-2536.)

An individual nanocoax in either the fiber coupled or light emitting preparation can elicit physiologically relevant photocurrents in optogenetic acute brain slices. For example, one can record from pyramidal neurons from acute brain slices. (Varela, et al. 2012 J. Neurosci. 32:12848-12853.) For optical control of neurons, the neuron silencing inhibitory halorhodopsin (eNpHr3.0) or the neuron-exciting channel rhodopsin 2 (ChR2) are transduced via adenoassociated (serotype 5) viral constructs containing the optogenetic material (eNpHr3.0 or ChR2) and a fluorescent tag (mCherry or eYFP), or only the fluorescent tag as an experimental control all under the CamKIIa promoter to selectively target pyramidal neurons.

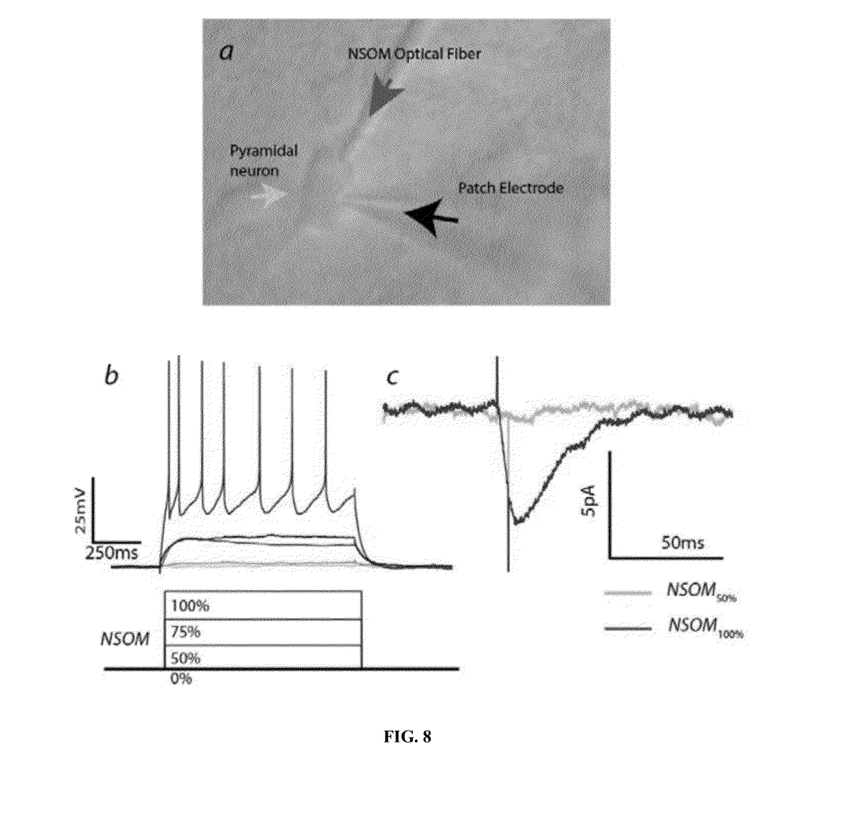

For light sensitive brain slices, the virus is injected to the various regions of interest, including amygdala, sensory cortex and hippocampus, under surgical anesthesia several days prior to tissue collection using established protocols. (Varela, et al. 2012 J. Neurosci. 32:12848-12853.) A specially prepared optically transmitting nanocoax (as in FIG. 2) will be coupled to either blue (ChR2) or green (eNpHr3.0) 150 mW lasers and inserted into the recording chamber to provide light directly to the tissue under study (FIG. 8) and positioned adjacent to a ChR2 positive neuron (verified by eYFP fluorescence).

FIG. 8a shows infrared differential interference contrast image (IR-DIC) of pyramidal neuron (yellow arrow) in a whole cell patch configuration (black arrow). The tissue was obtained from rats 3 weeks after transfection with the channel rhodopsin 2 viral vector (AAV5-CaMKIIa-hChR2(H134R)-EYFP). An NSOM probe (blue arrow, as in FIG. 5) was positioned just above the cell membrane. The tip of the NSOM probe is <300 nm, too small to discern in these optical conditions. When configured as a nanocoax, light can be isolated only to the NSOM tip. In FIG. 8b, a voltage trace shows depolarization to 50 pA current injection (black trace) or 473 nm blue light administered through the NSOM probe at increasing power: 0, 50, 75 or 100% laser power (blue shaded traces), recorded with kynurenic acid (glutamate receptor antagonist) and SR95531 (gabazine, GABAa receptor antagonist) in the bath to prevent synaptic responses. Note the more rapid activation of ChR2 channels in response to light (leftward shift of blue trace) than to current injection. In FIG. 8c, after wash out of kynurenic acid and gabazine, 5 ms light “injections” evoked postsynaptic response akin to glutamate mediated AMPA currents.

Nanocoax wires configured as optrodes with the inner core as a recording electrode and the outer shield as ground and positioned near the surface of a ChR2 positive neuron can be fabricated with optimal desired characteristics, as shown in FIG. 8. A whole cell recording can be made from a conventional patch pipette. Injection of current via patch evokes action potentials in the recorded cell, which can be detected as an extracellular action potential by the optrode. A 473 nm blue light (power about 10 mW/mm²) delivered through the fiber optic depolarizes the patched cell and trains should evoke action potentials (spikes) detectable by the optrode. Conversely, blue light delivered by the optrode should elicit photocurrents in the patch cell that vary as a function of output intensity and wavelength measured concurrently by extracellular (via optrode) and intracellular (via patch) sensors. NCOAs, as depicted in FIG. 6, can be used to probe neurologic function of optogenetically prepared neurons in brain tissue.

### Nanocoax Optrode Arrays (NCOA) for Optogenetic Electrophysiology

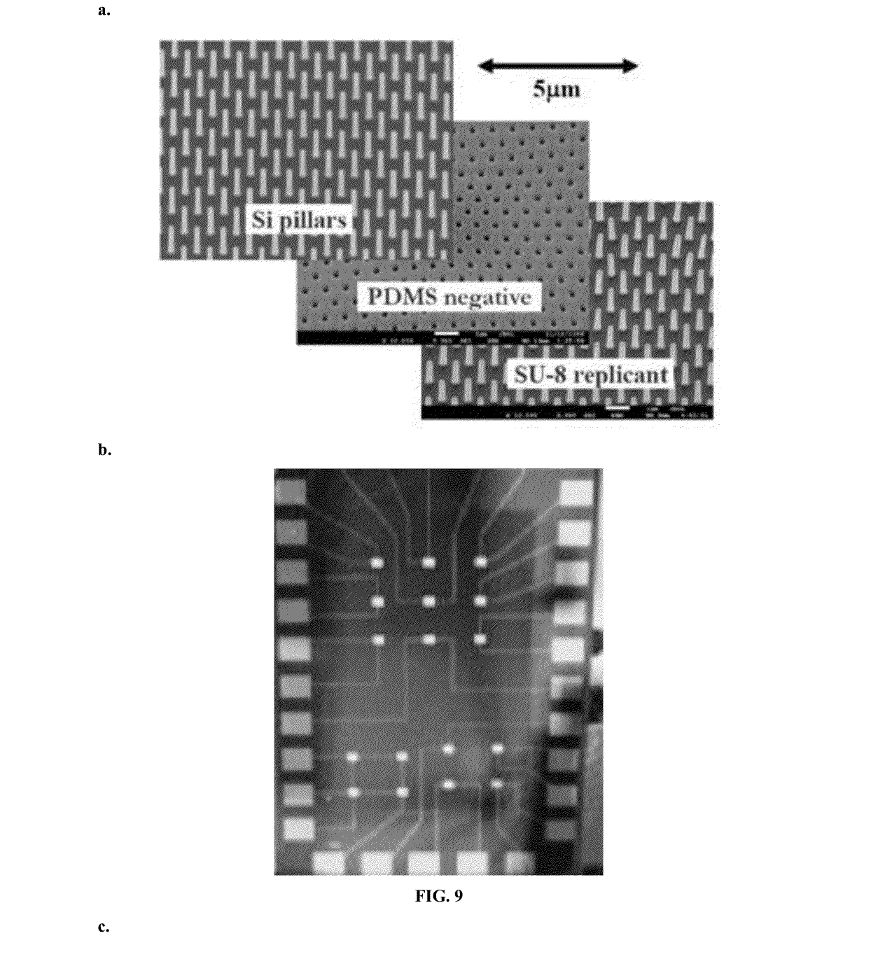

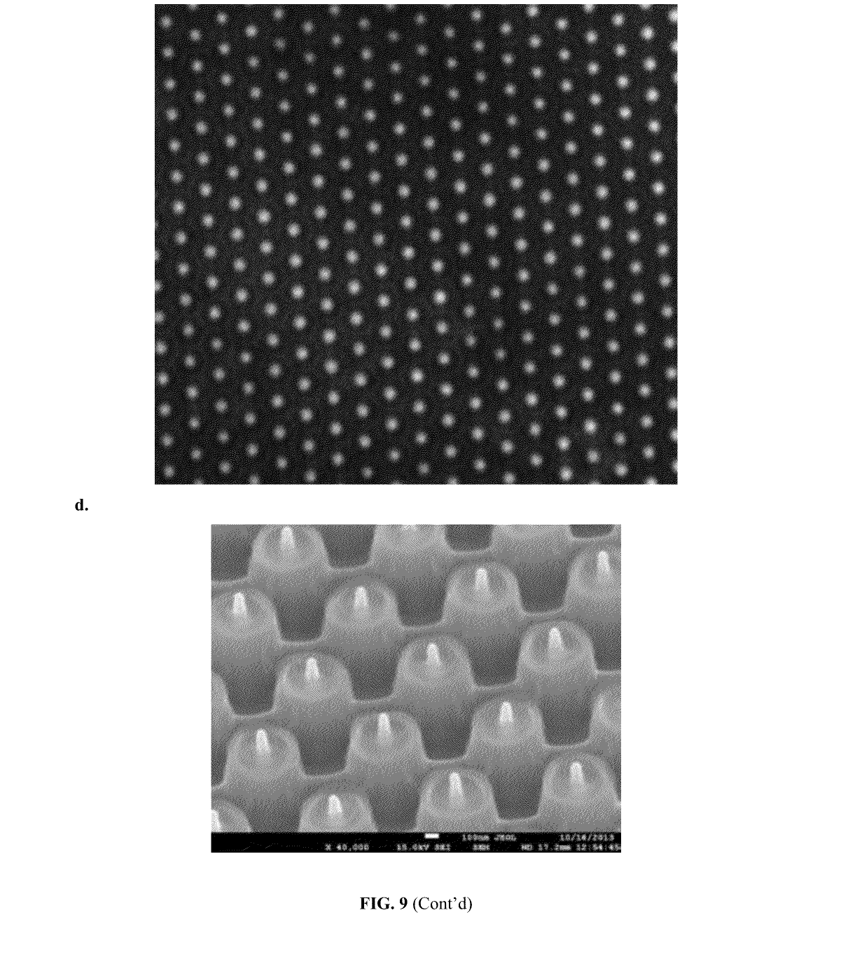

As shown in FIG. 9, the initial arrays can be fabricated as discrete subarrays (i.e. groups of nanocoaxes wired and thus operating in parallel), with inter-subarray spacings large enough to ensure independent electrical addressability. FIG. 9a depicts a NIL nanopillar fabrication process: SEM images of master array of 200 nm-diameter, 2 μm-tall Si nanopillars, an imprinted negative stamp, and an SU-8 polymer replica of the master. FIG. 9b is a photograph of a 10×20 mm² chip with 9 subarrays of nanocoaxes wired as differential addressable and 8 in a network configuration. Each measures 500 μm on a side.

FIG. 9c is an optical micrograph of back-illuminated, flexible nanocoax array (1.5 μm pitch), showing light has propagated through the annulus of each coax. An SEM image of an optically transmitting nanocoax-based array (NCOA) prepared with protruding cores is shown in FIG. 9d, which may facilitate cell immobilization and potential intracellular recording.

“Master” nanopillars can be fabricated via a variety of standard semiconductor lithography techniques. Alternatively, a novel, low-cost and high fidelity “nanoimprint lithography” (NIL) method can be employed to produce polymer replicas of Si nanopillars arrays. This process, illustrated in FIG. 9a, involves coating silicon pillars with a polymer (polydimethylsiloxane, PDMS) to make an elastomer “negative” stamp of the pillar array, which is then used to mechanically imprint the pattern into another polymer, resulting in a replica of the original array.

Control over the physical dimensions of these nanopillars, and thus the completed sensor device, with respect to height, diameter, and pitch, is important for optimizing NCOA device performance. Using these nanopillars, a common UV-curable polymer SU-8 can be used in a NIL apparatus for the replication process. The SU-8 replicates are then coated with three layers, metal-dielectric-metal, to form coaxial nanostructures. The tops of these nanocoaxes can be removed, using standard semiconductor industry wafer polishing techniques, to expose the inner electrode and dielectric in the coax annuli, the latter of which will be optically transmitting, similar to the NSOM-derived device above.

For the fabrication of electrically addressing individual arrays in a phased approach, devices can be prepared with subarrays having successively decreasing numbers of individual nanocoaxes each (at about 1 μm pitch), with subarrays separated by about 10 μm, as shown in FIG. 9b. An array containing N² nanocoaxes (i.e. an N×N array) requires 2N², N²+1 or 2N wires, depending on whether the electrical connections are differential, single-ended or network addressable, respectively. From a lithography and fabrication standpoint at about 1 μm to 2 μm array pitch, the network addressable architecture is the most desirable, as it requires the fewest wires (2N) to be fit into the structure wires. For example, nanocoax arrays at the 10×15 μm² scale containing about 100 networked coaxes have been produced.

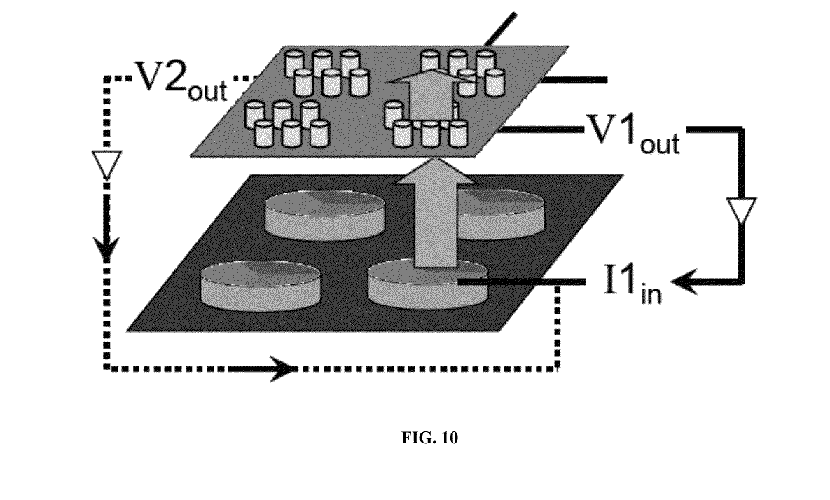

To make LED-coupled arrays, NCOA chips can be integrated with controllable LED arrays, as depicted in FIG. 10. FIG. 10 shows a schematic illustration of LED-coupled NCOA subarrays, spatially separated (about 10 μm scale) to allow electrically addressing using photolithographic techniques, and allowing validation of optical throughput and stimulation requirements when interfaced with optogenetic neurons. The NCOA can be mated with presently available LED arrays, facilitating discrete delivery of light to subarrays, with selectable wavelength (color) and intensity.

For example, commercially available LED arrays can be employed, and the NCOA fabrication can be adapted to prepare subarrays with size and spacing that matches that of the LED arrays. The display chips that are used for development purposes are inexpensive light emitting displays such as the organic light emitting diode (OLED) displays (e.g., small RGB capable OLED displays by Densitron). These displays can be interfaced to the controlling computer via a USB port.

The light emitting nanocoax (LENC) array has a major advantage over the LED-coupled array, for example, in that control of the light output and electrical recording can be configured on the exact same hardware scheme.

For in vitro testing of NCOA as a functional electrode array, a standard hippocampal brain slice preparation can be used. The anatomical organization of the hippocampus has made it a useful preparation for the study of synaptic plasticity. (Malenka, et al. 2004 Neuron 44:5-21.) Acute ChR2 expressing hippocampal slices are placed on the NCOA. A conventional stimulating electrode and macroscopic optical fiber is positioned in the Schaffer collateral and a conventional extracellular recording electrode placed in the stratum pyramidale. The observations are: (1) Electrically evoked or optically evoked excitation in the Shaffer collateral elicits field excitatory post synaptic potentials (fEPSP) in the stratum pyramidal recorded by the conventional extracellular electrode and by optrode clusters on the NCOA. (2) Brief blue-light pulses delivered to the brain slice through the NCOA, specifically to the pixels underlying the Schaffer collateral to elicit fEPSPs that scale in amplitude with light power.

### Variable Depth NCOAs for Volumetric Optogenetic Integration

The fabrication process for NCOAs may use variable length wires, which are both (1) fine enough to be minimally invasive as neural implants, and (2) mechanically robust at significant recording depths (>100s of μm). Current optogenetic viral vectors allow one to introduce membrane bound ion channels (or pumps) to phenotypically distinct neuron types, in anatomically specific pathways. For example, the insular cortex receives sensory inputs from both thalamic and sensory cortex. To control these pathways independently, one can introduce opsins to thalamic or sensory cortical regions with distinct excitation spectra (e.g., blue light or red-light shifted variants of ChR2), respectively. These inputs likely form synaptic contacts on distinct and common neurons in the insular cortex. A 3D NCOA that penetrates the insula, with electrodes and light guides capable of blue or red light excitation can be used to functionally map the thalamic and sensory cortex terminal fields over a significant volume of tissue and subsequently interrogate their function in a variety of ways.

Existing current optrode configurations are not scalable to volumetric, closed loop control. The invention herein advances the 2D NCOA fabrication process to 3D. The “bed of nails” array fabricated for chronic implantation may be fabricated. By relaxing the spacing of electrodes in the x, y plane, individual nanocoax wires can extend at variable lengths into the z plane. This approach is akin to the “Utah” arrays or commercially available floating microelectrode arrays (Plexon) but at a far smaller pitch and with integrated optical functionality. (Jones, et al. 1992 Ann. Biomed. Eng. 20:423-37.)

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A nanocoaxial electrode, comprising:
a conductive inner core;
a coaxial dielectric layer surrounding the conductive inner core; and
a coaxial conductive outer layer encasing the dielectric layer,
wherein
the coaxial dielectric layer is adapted to electrically isolate the conductive core from the conductive outer layer, and
the diameter of the coaxial conductive outer layer is less than about 2 μm.

2. The nanocoaxial electrode of claim 1, wherein the conductive inner core and conductive outer layer are adapted to serve as recording and reference electrodes.

3. The nanocoaxial electrode of claim 1, wherein the coaxial dielectric layer is adapted to serve as an optical light guide.

4. The nanocoaxial electrode of claim 1, wherein the coaxial dielectric layer is adapted to serve as a light emitting diode providing spatially discrete illumination.

5. The nanocoaxial electrode of claim 1, wherein the coaxial dielectric layer is adapted to serve as an electroluminescent component providing spatially discrete illumination.

6. The nanocoaxial electrode of claim 1, further comprising an embedded metal nanowire optical antenna.

7. The nanocoaxial electrode of claim 1, wherein the conductive inner core is made from a material selected from Ag, Au, C—Ga, W—Ga, Ni, Cr, Ti, Al and IrOx.

8. The nanocoaxial electrode of claim 1, wherein the dielectric layer is made from a material selected from Al₂O₃, SiO₂ and SU-8.

9. The nanocoaxial electrode of claim 1, wherein the conductive outer layer is made from a material selected from Ag, Au, Cr, Ti, Al, Pt, C, W and Ni.

10. The nanocoaxial electrode of claim 1, wherein the diameter of the coaxial conductive outer layer is less than about 1 μm.

11. The nanocoaxial electrode of claim 10, wherein the diameter of the coaxial conductive outer layer is less than about 500 nm.

12. A nanocoaxial optrode array, comprising:
a plurality of inter-connected nanocoaxial electrodes arranged in an array;
a light delivery component; and
an electronic recording component,
wherein the nanocoaxial electrode comprises a conductive inner core; a coaxial dielectric layer surrounding the conductive inner core; and a coaxial conductive outer layer encasing the dielectric layer, and wherein the diameter of the coaxial conductive outer layer is less than about 2 μm.

13. The nanocoaxial optrode array of claim 12, wherein the nanocoaxial optrode array is coupled to a light emitting diode (LED) array.

14. The nanocoaxial optrode array of claim 12, wherein the nanocoaxial electrodes have a protruding inner conductive core.

15. The nanocoaxial optrode array of claim 12, wherein the coaxial dielectric layer of nanocoaxial electrodes is adapted to serve as an optical light guide.

16. The nanocoaxial optrode array of claim 12, wherein the coaxial dielectric layer of nanocoaxial electrodes is adapted to serve as a light emitting diode.

17. The nanocoaxial optrode array of claim 12, wherein the coaxial dielectric layer of nanocoaxial electrodes is adapted to serve as an electroluminescent component providing spatially discrete illumination.

18. The nanocoaxial optrode array of claim 12, wherein the nanocoaxial electrode further comprising an embedded metal nanowire optical antenna.

19. A method for detecting extracellular neuronal activity, comprising:
providing a neuroelectronic probe comprising one or more nanocoaxial optrode arrays;
contacting the neuroelectronic probe with a tissue sample; and
manipulating the neuroelectronic probe to detect extracellular neuronal activity of the tissue sample.

20. The method of claim 19, further comprising recording one or more extracellular neuronal activities of the tissue sample.

21. The method of claim 19, further comprising recording one or more neurotransmitter molecular activities.

22. The method of claim 19, performed in vitro.

23. The method of claim 19, performed in vivo.
